(19) United States
(12) Patent Application Publication
(10) Pub. No.: US 2025/0276191 A1
(43) Pub. Date: Sep. 4, 2025

(54) ACTIVE MEDICAL DEVICE HAVING A LOW-PROFILE SOCKET HEADER FOR CONNECTION TO A LEAD

(71) Applicant: Greatbatch Ltd., Clarence, NY (US)

(72) Inventor: Christopher S. Mauhar, Maple Grove, MN (US)

(21) Appl. No.: 19/068,292

(22) Filed: Mar. 3, 2025

Related U.S. Application Data
(60) Provisional application No. 63/559,236, filed on Feb. 29, 2024.

(51) Int. Cl. A61N 1/375; A61N 1/378

(52) U.S. Cl. CPC A61N 1/3754; A61N 1/3758; A61N 1/378

(57) ABSTRACT

An (AMD) assembly has a device housing closed by a lid. A PCB assembly in the device housing is connected to a power source. A feedthrough connected to the lid has active terminal pins brazed into respective via holes in an insulator. A polymeric cover connected to the device housing has an axial recess extending along a cover lower surface. The proximal electrical contact portion of a lead comprises an M number of polymeric carriers, each supporting an electrical contact and contacting an N number of lead insulators in an alternating sequence extending distally from a proximal lead end. With the lead proximal electrical contact portion received in the cover axial recess and the cover received in a lid recess, an electrical contact supported by a carrier is connected to an active terminal pin connected to the PCB assembly and the electrical contact is connected to a lead electrode.

### CROSS-REFERENCE TO RELATED APPLICATION

This application claims priority to U.S. Provisional Application Ser. No. 63/559,236, filed on Feb. 29, 2024.

### BACKGROUND OF THE INVENTION

### 1. Field of the Invention

The present invention relates to the field of medical devices. More particularly, the present invention relates to an implantable or externally worn active medical device (AMD) that is designed to deliver electrical stimulation to a patient, sense biological signals from body tissue, or both send pulses and sense biological signals. In one embodiment, the AMD is a miniature-sized medical device. The AMD can be implanted in a patient's body or worn externally on the body.

The desire to make AMDs as small as possible is an active area of innovation. Implanting a miniature-sized AMD is advantageous over implanting a conventionally-sized pulse generator for many reasons. Chief among them is that the implantation procedure can be performed with far less surgical trauma to the patient. As long as the miniature AMD has the same or similar functionality as an AMD of a conventional size, subjecting the patient to less trauma represents an advancement in the industry. This includes implanting a miniature-sized neurostimulator for pain therapy. Additionally, a miniature-sized neurostimulator can be applied to many more nerves, particularly to smaller nerves, than a relatively larger conventionally-sized neurostimulator. Further, an externally worn miniature AMD would be expected to be less bothersome to a patient than a larger version of the same device.

### 2. Prior Art

A conventional AMD has a molded or cast header as a polymeric body connected to the medical device at its lid. The molded header as an assembly supports a number of terminal housings that are configured to detachably connect to the proximal electrical contact portion of a lead. A typical lead has at least two spaced-apart distal electrodes and a similar number of proximal electrical contacts. The distal electrodes are configured to send electrical pulses to the surrounding body tissue, sense biological signals from the tissue, or both send pulses and sense biological signals.

However, there are size limitations regarding how small the industry can miniaturize an active medical device that has a molded header connected to the device lid and that supports a number of terminal housings. The molded header adds bulk that can substantially increase the overall size of the medical device. In that respect, efforts to miniaturize AMDs are focused on integrating the active medical device with the lead into a single device. Although this simplifies connection between the medical device and its pacing/sensing lead, such medical devices cannot be customized according to the physical characteristics of the implantation procedure or the patient's medical condition.

Therefore, there is an ongoing need for an improved miniature-sized AMD, whether implantable or intended to be worn externally, that is detachably connectable to a lead to provide both stimulation and sensing capability. Instead of a molded or cast header supporting a plurality of terminal housings, the miniature-sized medical device has a feedthrough with a high density of active terminal pins extending into an interior of the device housing. The feedthrough active terminal pins are detachably connectable to specially designed electrical contacts at a proximal contact portion of a stimulation or sensing lead. A smaller medical device is easier to implant in a patient and is expected to cause less trauma to the patient. A smaller medical device is also expected to be less bothersome to a patient.

### SUMMARY OF THE INVENTION

In a general sense, the present invention relates to an active medical device having a device housing comprising a main housing closed by a lid. The main housing has a housing sidewall extending to an annular edge surrounding a housing opening leading into an interior of the main housing. The lid supports a feedthrough that is recessed below an upper edge of the lid. The feedthrough comprises a ferrule that is hermetically sealed to an oval-shaped ceramic insulator. The insulator supports a first plurality of active terminal pins and a second plurality of non-active terminal pins. Each active terminal pin has a proximal end extending inside the main housing where it is connected to electronic circuits or electronic components supported on a printed circuit board (PCB) as a PCB assembly. The PCB assembly is powered by an electrical power source contained inside the device housing. The distal ends of the active terminal pins extend outwardly beyond the ceramic insulator. The number of non-active terminal pins is less than the first plurality of active terminal pins, preferably half as many, but that is not necessary. The non-active terminal pins, which do not extend completely through the ceramic insulator, also have distal ends that extend outwardly beyond the insulator.

A preferred pattern for the active terminal pins and non-active terminal pins extending along the oval-shaped ceramic insulator of the feedthrough comprises an active terminal pin followed by an active terminal pin/non-active terminal pin pair, followed by an active terminal pin, followed by another active terminal pin/non-active terminal pin pair. This sequence continues axially along the ceramic insulator for as many active terminal and active terminal pin/non-active terminal pin units as are required for the medical condition that the AMD is designed to treat. In a preferred embodiment, there are two axial rows, each comprising a sequence of a unit comprising an active terminal pin and an active terminal pin/non-active terminal pin pair arranged side-by-side along the length of the ceramic insulator.

A specially designed lead has a proximal electrical contact portion that comprise an alternating series of a plurality of rigid polymeric tubular socket carriers connected to an electrical contact, and a plurality of ring-shaped insulators. The tubular socket carriers and the ring-shaped polymeric insulators alternate one then the other from a proximal-most tubular socket carrier to a distal-most insulator ring, both supported on the semi-rigid lead body.

In use, with the proximal electrical contact portion of the lead received in the axial recess of the cover and with the cover received in the lid recess, the electrical contacts supported by a respective carrier are electrically connected to a respective one of the active terminal pins of the feedthrough to thereby establish electrical continuity from the PCB assembly connected to the active terminal pins in turn connected to a respective electrical contact connected to a respective first electrical conductor connected to a distal electrode of the lead. The lead is configured to at least one of deliver electrical stimulation to body tissue or sense biological signals from body tissue.

More particularly, the present invention relates to an active medical device (AMD) assembly comprising a device housing closed by a lid having an upper surface and a lid opening. A printed circuit board (PCB) assembly contained in the device housing is powered by an electrical power source. A feedthrough includes a ferrule having a ferrule upper surface and a ferrule opening. An insulator is brazed into the ferrule opening and a plurality of first active terminal pins are brazed into a respective one of a plurality of first via holes extending through the insulator. Then, the feedthrough is connected to the lid in the lid opening so that the ferrule upper surface is spaced below a lid upper surface to provide a lid recess.

A polymeric cover that is detachably connectable to the device housing comprises a main cover portion having a cover lower surface. A first axial recess extends along the cover lower surface.

A first lead extends distally from a first proximal electrical contact portion to at least two first distal electrodes configured to contact body tissue. The first proximal electrical contact portion comprises a first M number of polymeric carriers, each supporting a first electrical contact, and contacting a first N number of lead insulators in an alternating sequence extending distally from a proximal end of the first proximal electrical contact portion of the first lead. The alternating, distally extending sequence comprises a first of the first N number of lead insulators contacting a first of the first M number of carriers supporting a first electrical contact, a second of the first N number of lead insulators contacting the first of the first M number of carriers supporting a first electrical contact, a second of the first M number of carriers supporting a first electrical contact and contacting the second of the first N number of lead insulators, a third of the first N number of lead insulators contacting the second of the first M number of carriers supporting a first electrical contact, a third of the first M number of carriers supporting a first electrical contact and contacting the third of the first N number of lead insulators, a fourth of the first N number of lead insulators contacting the third of the first M number of carriers supporting a first electrical contact, a fourth of the first M number of carriers supporting a first electrical contact and contacting the fourth of the first N number of lead insulators, a fifth of the first N number of lead insulators contacting the fourth of the first M number of carriers supporting a first electrical contact and continuing distally to a distal-most one of the first M number of carriers supporting a first electrical contact and distally contacting a distal-most one of the first N number of lead insulators.

In that manner, each of the first electrical contacts supported by a respective one of the first M number of carriers is connected to a first electrical conductor that extends to a first distal electrode of the first lead. Then, with the first proximal electrical contact portion of the first lead received in the first axial recess of the cover and with the cover received in the lid recess, the first electrical contacts supported by the respective first M number of carriers are electrically connected to a respective one of the first active terminal pins which are connected to the PCB assembly. The first electrical contacts connected to a respective first electrical conductor connected to a distal electrode of the first lead.

In one embodiment, the first electrical contacts supported by a respective one of the first M number of carriers have an active opening that receives a respective first active terminal pin to thereby establish electrical continuity from the PCB assembly to the first electrical contacts.

The AMD assembly of the present invention further includes a plurality of second active terminal pins that are brazed into a respective one of a plurality of second via holes aligned side-by-side with the first active terminal pin in the first via holes in the feedthrough insulator. The cover further comprises a second axial recess extending along the cover lower surface with the first and second axial recesses being aligned side-by-side in the cover lower surface.

A second lead extends distally from a second proximal electrical contact portion to at least two second distal electrodes configured to contact body tissue. The second proximal electrical contact portion comprises a second M number of polymeric carriers supporting a respective second electrical contact and contacting a second N number of lead insulators in an alternating sequence extending distally from a proximal end of the second proximal electrical contact portion of the second lead. The alternating, distally extending sequence comprises a first of the second N number of lead insulators contacting a first of the second M number of carriers supporting a second electrical contact, a second of the second N number of lead insulators contacting the first of the second M number of carriers supporting a second electrical contact, a second of the second M number of carriers supporting a second electrical contact and contacting the second of the second N number of lead insulators, a third of the second N number of lead insulators contacting the second of the second M number of carriers supporting a second electrical contact, a third of the second M number of carriers supporting a second electrical contact and contacting the third of the second N number of lead insulators, a fourth of the second N number of lead insulators contacting the third of the second M number of carriers supporting a second electrical contact, a fourth of the second M number of carriers supporting a second electrical contact and contacting the fourth of the second N number of lead insulators, a fifth of the second N number of lead insulators contacting the fourth of the second M number of carriers supporting a second electrical contact and continuing distally to a distal-most one of the second M number of carriers supporting a second electrical contact and distally contacting a distal-most one of the second N number of lead insulators.

In that manner, each of the second electrical contacts supported by a respective one of the second M number of carriers is connected to a second electrical conductor that extends to a second distal electrode of the second lead. Then, with the second proximal electrical contact portion of the second lead received in the second axial recess of the cover and with the cover received in the lid recess, the second electrical contacts supported by the respective second M number of carriers are electrically connected to a respective one of the second active terminal pins of the feedthrough to thereby establish electrical continuity from the PCB assembly connected to the second active terminal pins connected to the second electrical contacts connected to a respective second electrical conductor connected to a second distal electrode of the second lead.

The AMD assembly of the present invention further has a plurality of non-active terminal pins brazed into a respective one of a plurality of first non-active via holes in the feedthrough insulator. The first electrical contacts supported by the second, fourth, and continuing distally for every even number of the first M number of carriers also has a non-active opening which receives a respective first non-active terminal pin, or the first electrical contacts supported by the first, third, and continuing distally for every odd number of the first M number of carriers also has a non-active opening which receives a respective first non-active terminal pin.

The AMD assembly of the present invention further comprises the main cover portion extending to a depending cover portion with the main cover portion extending along a primary cover axis from a distal cover end wall to the depending proximal cover portion aligned along a secondary cover axis that is substantially perpendicular to the primary cover axis. The depending proximal cover portion is disposed adjacent to the housing sidewall. Further, the first and second axial recesses extending along the cover lower surface extend through the depending proximal cover portion to an outer surface of the depending cover portion.

The AMD assembly of the present invention further has the main cover portion extending to the depending cover portion comprising a cover upper surface spaced from a cover lower surface with the cover upper and lower surfaces extending to a cover front sidewall opposite a cover back sidewall. An endless groove residing in the front and back sidewalls, the curved distal end wall and the depending proximal curved portion supports a pliable endless ring-shaped sealing gasket.

The AMD assembly of the present invention further has the plurality of non-active terminal pins brazed into a respective one of the plurality of first non-active via holes not extending through the insulator thickness into the interior of the main housing.

The AMD assembly of the present invention further comprises the plurality of first active terminal pins having a first diameter perpendicular to their first longitudinal axis and the plurality of first non-active terminal pins having a second diameter perpendicular to their second longitudinal axis, and the second diameter of the first non-active terminal pins is equal to, less than or greater than the first diameter of the first active terminal pins.

The AMD assembly of the present invention further provides the ferrule opening having an oval-shaped comprising a proximal curved end opposite a distal curved end. The ferrule of the feedthrough has a threaded opening adjacent to the proximal curved end and an outwardly extending locking tab adjacent to the distal curved end of the oval-shaped ferrule opening. The cover has a threaded opening adjacent to the distal cover end wall and a locking recess located in the depending proximal cover portion. In that manner, when the cover is detachably connected to the device housing, the outwardly extending locking tab of the ferrule is received in the locking recess in the depending proximal cover portion and the cover threaded opening is aligned with the ferrule threaded opening so that a fastener threaded through the cover threaded opening aligned with the feedthrough threaded opening connects the cover to the device housing.

These and other aspects of the present invention will become increasingly more apparent to those skilled in the art by reference to the following detailed description and to the appended drawings.

### BRIEF DESCRIPTION OF THE DRAWINGS

FIG. 1 is a wire-formed diagram of a generic human body showing a number of medical devices 100A to 100L according to the present invention that can either be implanted in a patient's body tissue or attached externally to the body.

FIG. 2 is a simplified block diagram of an exemplary medical device system 10 according to the present invention.

FIG. 3 is a perspective view of an exemplary active medical device (AMD) 12 according to the present invention, which is any of various types of the AMDs that are shown in FIG. 1.

FIG. 4A is an exploded perspective view of the AMD 12 shown in FIG. 3 but now including a polymeric cover 54 that fits into the medical device and a pair of side-by-side leads 200 that are configured to be received in the cover.

FIG. 4B is a schematic view showing side-by-side leads 200 according to the present invention, both extending from 202 proximal electrical contact portions 202 to distal electrodes 203A, 203B that are configured to deliver current pulses to body tissue, for example, the heart H in which the distal electrodes are implanted, receive sensed electrical signals pertaining to functions of the body tissue, or both sense electrical signals and deliver current pulses.

FIG. 5 is a side elevational view of the polymeric cover 54 shown in FIG. 4A.

FIG. 6 is a perspective view of the polymeric cover 54 shown in FIGS. 4A and 5 prior to receiving the side-by-side leads 200.

FIG. 7A is a perspective view of the lead 200 shown in FIGS. 4A and 6 prior to having its components over molded with a polymeric material.

FIG. 7B is a perspective view of the lead 200 shown in FIG. 7A after having its components over molded with a polymeric material 222.

FIG. 8 is a perspective view looking at the underside of the polymeric cover 54 after having received the proximal electrical contact portions 202 of the two side-by-side leads 200.

FIG. 9 is a plan view looking at the underside of the polymeric cover 54 shown in FIG. 8 after having received the proximal electrical contact portions 202 of the two side-by-side leads 200.

### DETAILED DESCRIPTION OF THE PREFERRED EMBODIMENTS

As used herein, the term “active medical device” means a medical device, whether implantable or worn externally, that is designed to deliver electrical stimulation to a patient, sense biological signals from body tissue, or both stimulate and sense.

The term “active terminal pin” means a terminal pin that is brazed into the insulator of a feedthrough for an active medical device. The active terminal pin has an active terminal pin device portion extending into the interior of a medical device housing where the terminal pin is connected to the electronic component of a PCB assembly and has an active terminal pin body fluid portion extending outwardly beyond the body fluid side of the insulator. The active terminal pin is configured to have electrical stimulation pulses and biological signals travel along its length between the PCB assembly and an electrical contact for the medical device.

The term “non-active terminal pin” means a terminal pin that is brazed into the insulator of a feedthrough for an active medical device, but its device side portion may or may not extend into the interior of a medical device housing. If the device side portion of a non-active terminal pin extends into the interior of the medical device housing, it is not connected to the PCB assembly. However, the non-active terminal pin has a non-active terminal pin body fluid portion that extends outwardly beyond the body fluid side of the insulator.

The term “via hole” means an opening that either extends completely through the thickness of a feedthrough insulator from an insulator device side facing an interior of a device housing to an opposed insulator body fluid side, or the opening extends from the insulator body fluid side at least part-way through the insulator thickness.

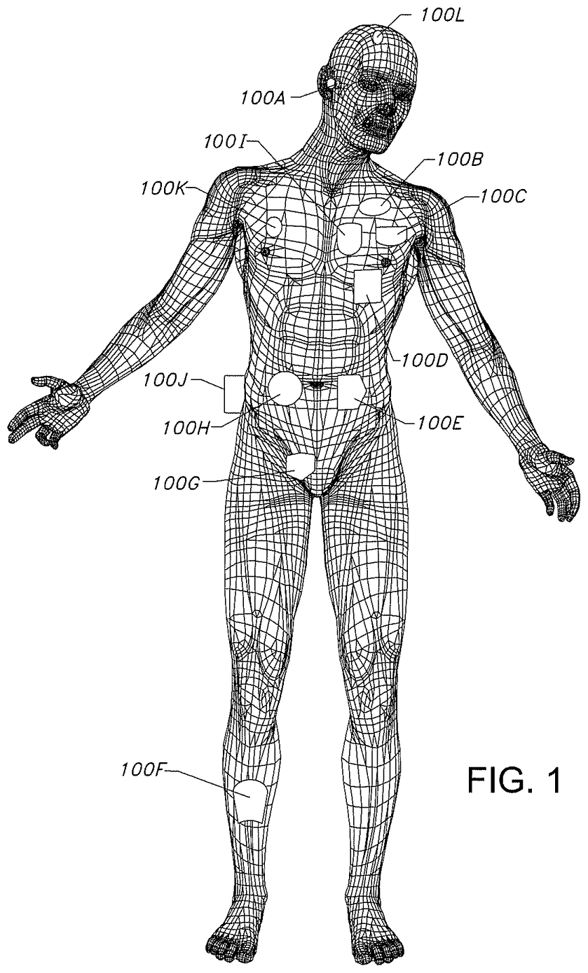

Turning now to the drawings, FIG. 1 is a wire form diagram of a generic human body illustrating various types of active implantable and external medical devices according to the present invention that can either be implanted in a patient's body or attached externally to the body.

Numerical designation 100A represents a family of hearing devices which can include the group of cochlear implants, piezoelectric sound bridge transducers, and the like.

Numerical designation 100B represents a variety of neurostimulators, brain stimulators, and sensors. Neurostimulators are used to stimulate the Vagus nerve, for example, to treat epilepsy, obesity, and depression. Brain stimulators are pacemaker-like devices and include electrodes implanted deep into the brain for sensing the onset of a seizure and also providing electrical stimulation to brain tissue to prevent a seizure from actually occurring. The lead wires associated with a deep brain stimulator are often placed using real time MRI imaging. Sensors include optical sensors, motion sensors, acoustic sensors, pressure sensors, analyte sensors, and electromagnetic sensors, among others.

Numerical designation 100C shows a cardiac pacemaker which is well-known in the art.

Numerical designation 100D includes the family of left ventricular assist devices (LVADs), and artificial heart devices.

Numerical designation 100E includes a family of drug pumps which can be used for dispensing insulin, chemotherapy drugs, pain medications, and the like.

Numerical designation 100F includes a variety of bone growth stimulators for rapid healing of fractures.

Numerical designation 100G includes urinary incontinence devices.

Numerical designation 100H includes the family of pain relief spinal cord stimulators and anti-tremor stimulators.

Numerical designation 100H also includes an entire family of other types of neurostimulators used to block pain.

Numerical designation 100I includes a family of implantable cardioverter defibrillator (ICD) devices and also the family of congestive heart failure devices (CHF). This is also known in the art as cardio resynchronization therapy devices, otherwise known as CRT devices.

Numerical designation 100J illustrates an externally worn pack. This pack could be an external insulin pump, an external drug pump, an external neurostimulator or even a ventricular assist device.

Numerical designation 100K illustrates one of various types of EKG/ECG external skin electrodes which can be placed at various locations on the patient's body.

Numerical designation 100L represents external EEG electrodes that are placed on the patient's head.

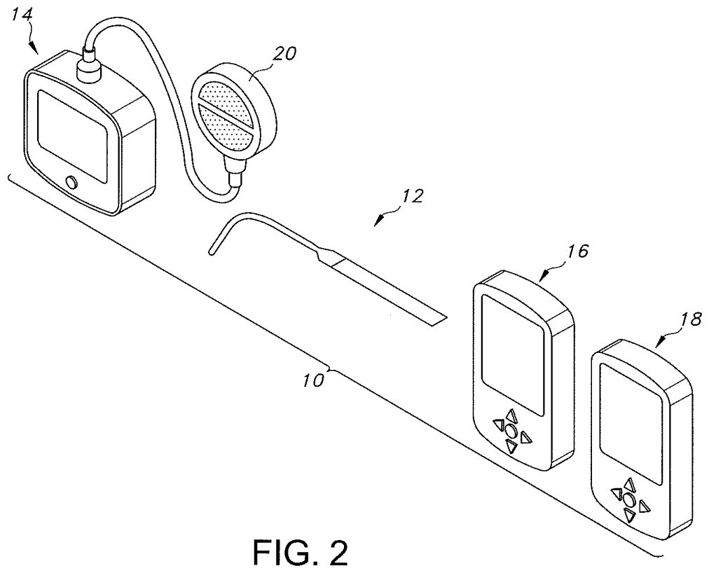

To provide context to the various medical devices 100A to 100L illustrated in FIG. 1, FIG. 2 illustrates a simplified block diagram of an exemplary medical device system 10 according to the present invention. The medical device system 10 includes an active medical device (AMD) 12, which represents any of the various types of medical devices that include a lead, whether implantable or external, that are described above with reference to FIG. 1. If implantable, the lead is configured to at least one of deliver electrical stimulation to body tissue or sense biological signals from body tissue. The medical device system 10 also has an external charger 14, a patient programmer 16, and a clinician programmer 18.

The patient programmer 16 and the clinician programmer 18 may be portable handheld devices, such as a smartphone or other custom device, that are used to configure the AMD 12 so that the AMD can operate in a desired manner. The patient programmer 16 is used by the patient in whom the AMD 12 is implanted. The patient may adjust the parameters of electrical stimulation delivered by the AMD 12, such as by selecting a stimulation program, changing the amplitude and frequency of the electrical stimulation, among other parameters, and by turning stimulation on and off. Additionally, the patient programmer 16 may collect and display data being collected by the device 12 and alert the patient to potential health risks.

The clinician programmer 18 is used by medical personnel to configure the other system components and to adjust stimulation parameters that the patient is not permitted to control. These include setting up stimulation programs among which the patient may choose and setting upper and lower limits for the patient's adjustments of amplitude, frequency, and other parameters. It is also understood that although FIG. 2 illustrates the patient programmer 16 and the clinician programmer 18 as two separate devices, they may be integrated into a single programmer in some embodiments.

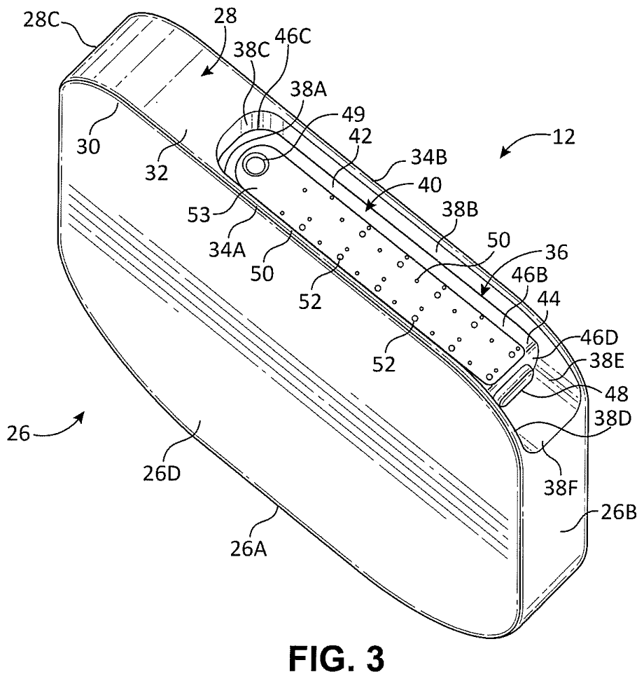

Electrical power can be delivered to the AMD 12 through an external charging pad 20 that is connected to the external charger 14. In some embodiments, the external charging pad 20 is configured to directly power the AMD 12 or it is configured to charge a rechargeable electrical power source 22 (FIG. 3) of the AMD. The external charging pad 20 can be a hand-held device that is connected to the external charger 14, or it can be an internal component of the external charger. The external charger 14 and the charging pad 20 can also be integrated into a single device that is strapped on or attached to the patient with adhesive, and the like.

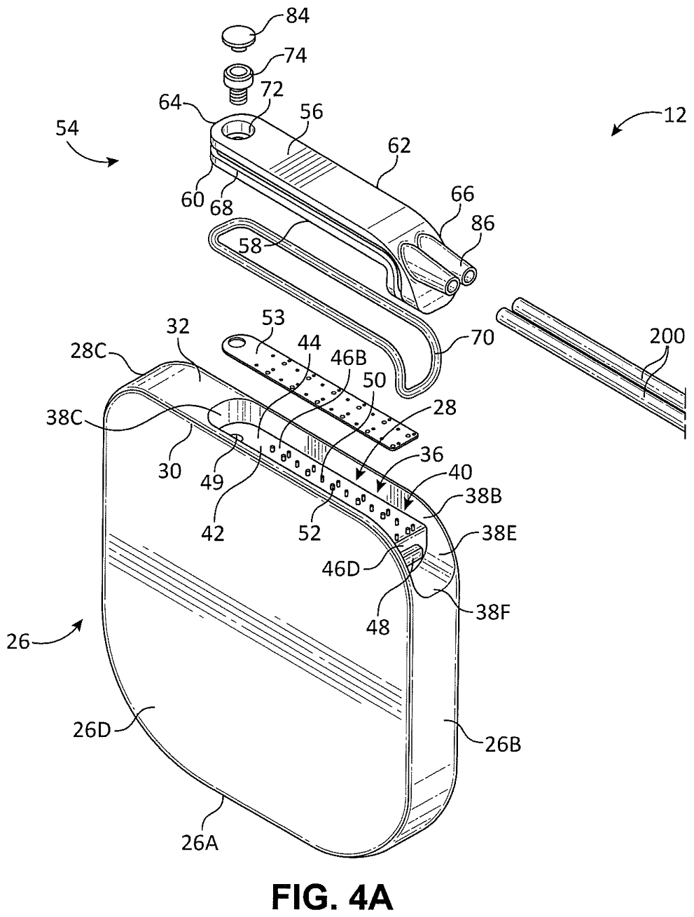

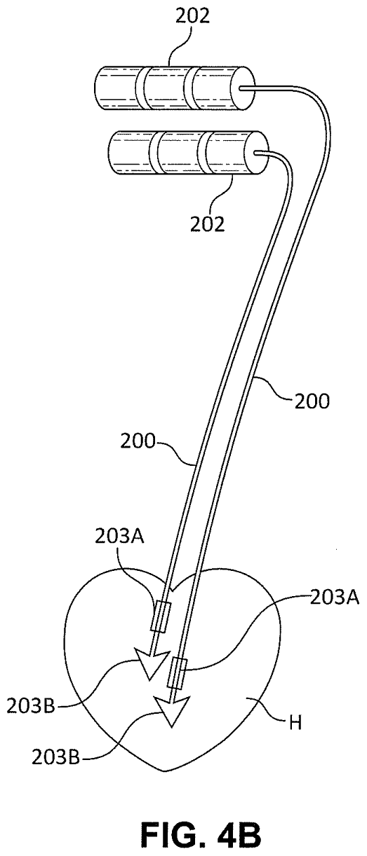

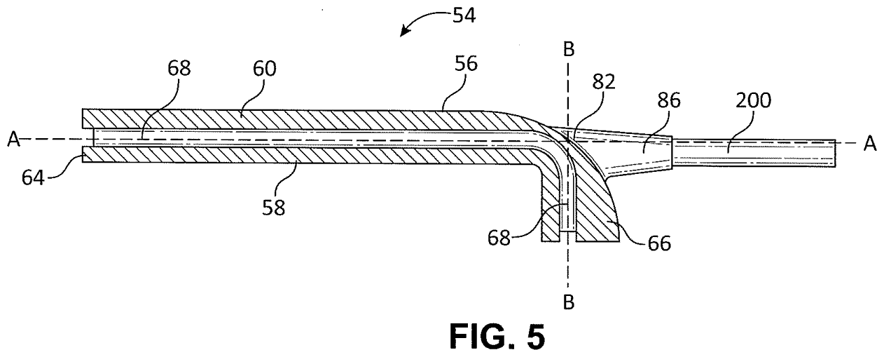

Referring now to FIGS. 3, 4A and 4B, these drawings illustrate the AMD 12 as an exemplary embodiment of the various medical devices 100A to 100L illustrated in FIG. 1 and the exemplary AMD 12 shown in the medical device system 10 in FIG. 2 that can be implanted in a patient's body or worn externally on a patient's body. The AMD 12 comprises a main housing 26 that is closed by a lid 28 to provide a device housing for the AMD. A printed circuit board (PCB) assembly (not shown) comprising a PCB supporting at least two electronic circuits or electronic components is housed inside the device housing. The device housing also contains an electrical power source (not shown) connected to the PCB assembly to provide electrical power to the at least two electronic circuits or electronic components. The PCB assembly in turn provides electrical power to leads 200 (FIGS. 4A, 4B, 5, 6, 7A, 7B, 8 and 9) that are detachably connected to the AMD 12. As is well understood by those skilled in the art, the leads 200 have a number of distal electrodes 203A, 203B (FIG. 4B) that are configured to deliver current pulses to body tissue, for example, the heart H in which the distal electrodes are implanted, receive sensed electrical signals pertaining to functions of the body tissue, or both sense electrical signals and deliver current pulses.

The electrical power source for the AMD 12 can be a capacitor or a rechargeable battery, for example, a hermetically sealed rechargeable Li-ion battery. However, the electrical power source is not limited to any one chemistry or even a rechargeable chemistry and can be of an alkaline cell, a primary lithium cell, a rechargeable lithium-ion cell, a Ni/cadmium cell, a Ni/metal hydride cell, a supercapacitor, a thin film solid-state cell, and the like. Preferably, the electrical power source is a lithium-ion electrochemical cell comprising a carbon-based or Li₄Ti₅O₁₂-based anode and a lithium metal oxide-based cathode, such as of LiCoO: or lithium nickel manganese cobalt oxide (LiNiₐMn_{b}Co_{1-a-b}O₂). The electrical power source can also be a solid-state thin film electrochemical cell having a lithium anode, a metal-oxide based cathode and a solid electrolyte, such as an electrolyte of LiPON (LiₓPO_{y}N_{z}).

The main housing 26 containing the PCB assembly and the electrical power source comprises a curved end wall 26A extending to opposed planar edge walls 26B and 28C and opposed planar face walls 26D (the face wall opposite wall 26D is not shown). Curved intermediate sidewalls connect to the opposed housing edge walls, to the opposed housing face walls, and to the housing bottom wall. In turn, the edge walls, face walls and curved intermediate sidewalls extend to an annular upper edge 30 surrounding an opening leading into an interior of the main housing 26. Titanium is a preferred material for the main housing 26.

The annular upper edge 30 of the main housing 26 is closed by the lid 28 comprising a generally planar upper wall 32 that resides between opposed curved edge walls 34A and 34B. The planar upper wall 32 and the curved edge walls 34A, 34B define an oval-shaped lid recess 36 that is bounded by a surrounding inner sidewall. The inner sidewall defining the lid recess 36 comprises opposed vertically-oriented inner sidewalls portions 38A and 38B that extend to a curved vertically-oriented distal inner sidewall portion 38C. Opposite the curved distal inner sidewall portion 38C, the inner sidewall portions 38A, 38B extend to depending opposed sidewall portions 38D and 38E that meet a lower sidewall portion 38F that faces upwardly. The lower sidewall portion 38F preferably has an upwardly-facing U-shape. The trough of the U-shaped lower sidewall portion 38F is spaced substantially below the generally planar upper wall 32 of the lid 28. Titanium is a preferred material for the lid 28.

A feedthrough 40 is received in the lid recess 36 spaced below the planar upper wall 32 of the lid 28. The feedthrough 40 comprises an oval-shaped ferrule 42 that is welded, preferably laser welded, into the oval-shaped lid recess 36. The ferrule 42 comprises a planar upper wall 44 extending laterally to opposed sidewalls 46B (the right sidewall opposite left sidewall 46B is not shown). The ferrule sidewalls 46B extend to a curved distal end wall 46C opposite a vertically-oriented proximal end wall 46D. A rectangularly-shaped locking tab 48 extends outwardly from the proximal end wall 46D. A threaded hole 49 resides adjacent to the curved distal inner sidewall portion 38C. The threaded hole 49 does not extend completely through the thickness of the ferrule 42.

An opening, preferably an oval-shaped opening (not shown) is centrally located in the ferrule 42. A ceramic insulator (not shown), for example, an oval-shaped alumina insulator, is brazed into the ferrule opening. The ceramic insulator has a thickness that extends from an insulator device side located inside the device housing to an opposed insulator body fluid side facing outwardly adjacent to the planar upper wall 32 of the lid 28. The feedthrough insulator has a first plurality of via holes (not shown) that extend through its thickness and a second plurality of via holes (not shown), preferably having a greater diameter than the first via holes, that extend through the thickness of the insulator, but if desired, are blind holes that do not extend completely through the insulator thickness. Titanium is a preferred material for the ferrule 42.

A first plurality of active terminal pins 50 are brazed into the first plurality of via holes and have a first diameter perpendicular to their longitudinal axis. The active terminal pins have their proximal ends connected to electronic components supported on the previously described PCB assembly (not shown) housed inside the medical device housing comprising the main housing 26 closed by the lid 28. A second plurality of non-active terminal pins 52 are brazed into the second plurality of via holes and have a second diameter perpendicular to their longitudinal axis. The second diameter of the non-active terminal pins is less than, equal to, or greater than, preferably greater than, the first diameter of the active terminal pins. The function of the non-active terminal pins 52 will be described in detail hereinafter. Brazing a ceramic insulator into a ferrule opening and brazing terminal pins, whether they are active terminal pins or non-active terminal pins, into via holes in an insulator is well known by those skilled in the feedthrough arts.

As shown in FIGS. 3 and 4A, the feedthrough 40 has two side-by-side rows comprising an active terminal pin 50 and an active terminal pin 50/non-active terminal pin 52 pair extending along the length of its insulator. In particular, each row begins with a first active terminal pin 50 that resides adjacent to the threaded hole 49 in the ferrule 42 and next to the curved distal inner sidewall portion 38C of the lid 28. Extending distally toward the vertically-oriented proximal end wall 46D of the ferrule 42, a side-by-side pair of a second active terminal pin 50/first non-active terminal pin 52 is supported by the feedthrough insulator. This is followed by a third active terminal pin 50, then a side-by-side pair of a fourth active terminal pin 50/second non-active terminal pin 52 pair. In total, there are six active terminal pins 50 followed by a side-by-side pair of an active terminal pin 50/non-active terminal pin 52. This sequence for both side-by-side rows ends with an active terminal pin 50/non-active terminal pin 52 pair comprising a twelfth active terminal pin 50 paired with a sixth non-active terminal pin 52 located next to the proximal end wall 46D of the ferrule 42. However, depending on the medical application that the AMD 12 is designed to treat, there can be less than or greater than that number of active terminal pins 50 followed by an active terminal pin 50/non-active terminal pin 52 pair as shown in the drawings.

If desired, a polymeric washer 53 having an appropriate hole pattern sized to receive the active terminal pins 50 and non-active terminal pins 52 is seated on the feedthrough insulator. The active terminal pins 50 and non-active terminal pins 52 extend upwardly beyond the upper surface of the washer 53.

In another embodiment of the present AMD, there is only one row of an active terminal pin 50 followed by an active terminal pin 50/non-active terminal pin 52 pair extending from adjacent to the threaded hole 49 in the ferrule 42 to adjacent to the proximal end wall 46D of the ferrule 42 for as many repeating units as needed.

Moreover, in another embodiment of the present AMD, the non-active terminal pins do not exist. Instead, there are two rows of side-by-side active terminal pins extending from adjacent to the threaded hole 49 in the ferrule 42 and next to the curved distal inner sidewall portion 38C of the lid 28 to adjacent to the proximal end wall 46D of the ferrule 42. Further, in still another embodiment according to the present invention, there is only one row of active terminal pins extending from adjacent to the threaded hole 49 in the ferrule 42 to adjacent to the proximal end wall 46D of the ferrule 42.

Referring now to FIGS. 4A, 5, 6, 8 and 9, the AMD 12 comprises a polymeric cover 54 that is sized and shaped to fit into the oval-shaped lid recess 36 formed by the planar upper wall 44 of the feedthrough ferrule 42 being spaced below the generally planar upper wall 32 and the opposed curved edge walls 34A and 34B of the lid 28. The cover 54 has a planar upper surface 56 spaced from a planar lower surface 58. The upper and lower surfaces 56, 58 extend to a front sidewall 60 opposite a back sidewall 62. The upper and lower surfaces 56, 58 meeting the front and back sidewalls 60, 62 extend to a distal end wall 64 opposite a depending proximal cover portion 66. Further, the cover 54 comprises a main cover portion extending along a primary cover axis A-A from the distal end wall 64 to the depending proximal cover portion 66 aligned along a secondary cover axis B-B. The secondary cover axis B-B is aligned substantially perpendicular to the primary cover axis A-A.

An endless groove 68 resides in the front and back sidewalls 60, 62, the curved distal end wall 64 and the depending proximal curved portion 66. A pliable endless ring-shaped sealing gasket 70 is seated in this groove 68. The sealing gasket 70 has a diameter that is greater than the depth of the groove 68 so that an outer portion of the gasket 70 extends outwardly beyond the perimeter of the polymeric cover 54. A pliable elastomeric material is preferred for the sealing gasket 70.

A threaded opening 72 extends through the upper and lower surfaces 56, 58 of the cover 54 adjacent to its distal end wall 64. A threaded fastener 74 is received in the opening 72.

The polymeric cover 54 also has a pair of side-by-side axial recesses 76 and 78 that extend proximally from a lead tip datum 80 residing adjacent to the threaded opening 72 to an inner end wall 82 of the depending proximal cover portion 66. The axial recesses 76, 78 extend through the depending proximal cover portion 66 to respective openings in a curved end wall 82 thereof.

A rectangularly-shaped recess 84 is located in the depending proximal cover portion 66 below the axial recesses 76, 78 and opposite the curved end wall 82. As will be described hereinafter, this recess 84 mates with the rectangularly-shaped locking tab 48 that extends outwardly from the proximal end wall 46D of the ferrule 42 with the feedthrough nested in the lid 28.

An integrated strain relief and boot seal 86 is supported by the polymeric cover 54 at its depending proximal portion 66. The strain relief and boot seal 86 has a pair of side-by-side openings that align with the pair of axial recesses 76, 78 in the polymeric cover 54.

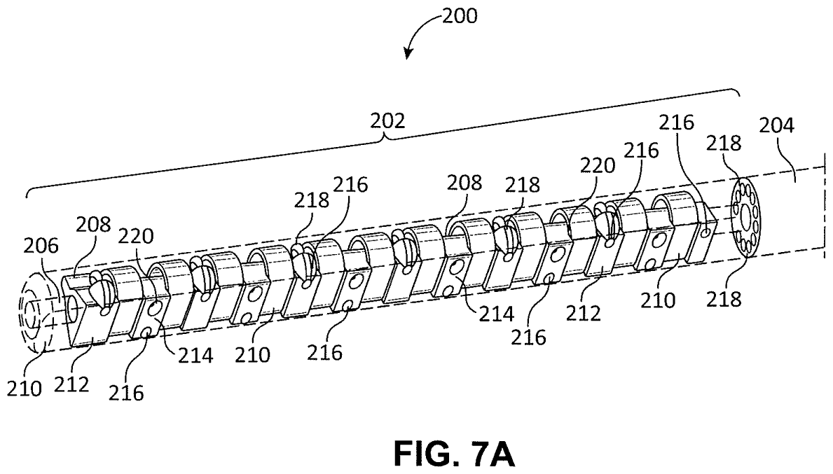

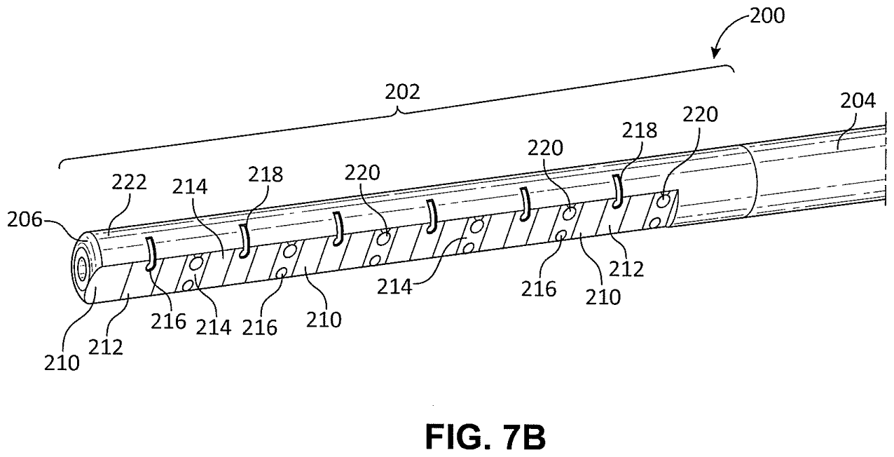

FIGS. 7A and 7B illustrate an implantable lead 200 according to the present invention. The illustrated leads 200 are substantially identical, although that is not required according to the present invention. If desired, the leads can have different characteristics such as length, number of electrodes, electrode configuration, and the like. The lead 200 illustrated in FIG. 7A is partially in phantom to show its interior construction. That is in contrast to the lead 200 illustrated in FIG. 7B, which is shown as it would appear in a surgical procedure. The leads 200 shown in FIGS. 7A and 7B comprise a novel proximal electrical contact portion 202 according to the present invention that is connected to a lead body 204. The lead body 204 extends distally to at least two distal electrodes 203A, 203B that are configured for contact with body tissue, for example, the heart H (FIG. 4B).

Turning now to the lead 200 shown in FIG. 7A, the proximal electrical contact portion 202 comprises a cylindrically-shaped, semi-rigid lead body 206 that supports an alternating series of a plurality of rigid polymeric tubular socket carriers 208 connected to an electrical contact, and a plurality of ring-shaped insulators 210. The tubular socket carriers 208 and the ring-shaped polymeric insulators 210 alternate one then the other from a proximal-most insulator 210 to a distal-most tubular socket carrier 208, and if desire, followed by a distal-most insulator ring 210 supported on the semi-rigid lead body 206. If desired, the cylindrically-shaped lead body 206 has a lumen that is sized to receive a stylet, a guidewire, and the like, or that provides for passage of fluids. In an alternate embodiment, the cylindrically-shaped, semi-rigid lead body 206 does not have a lumen. A thermally stabile rigid polymeric material is preferred for the pin socket carriers 208. The ring-shaped insulators 210 are preferably made from a thermos-plastic urethane.

Each tubular socket carrier 208 supports either a plate-shaped single-hole metallic electrical contact 212 or a plate-shaped two-hole metallic electrical contact 214. Looking at the proximal end of the lead 200 shown in FIG. 7A, a first tubular socket carrier 208 is connected to a first single-hole electrical contact 212 having an active hole 216 that is sized to receive a metallic tubular pin-socket (not shown). The proximal end of the tubular pin-socket received in the active hole 216 is then welded to the plate-shaped electrical contact 212 in a flush connection. The opposite end of the tubular pin-socket is crimped onto the proximal end of an electrical conductor 218 which extends distally along the lead to a corresponding electrode 203A, 203B that is configured for contact with body tissue, for example, the heart H (FIG. 4B).

Extending distally from the first tubular pin-socket carrier 208 connected to the first single-hole electrical contact 212, a first polymeric insulator ring 210 is positioned between the first carrier 208 and a second tubular pin-socket carrier 108 connected to a first plate-shaped two-hole electrical contact 214. In a similar manner as the single-hole contact 208, the two-hole electrical contact 214 has an active hole 216 that receives the proximal end of a tubular pin-socket (not shown). The tubular pin-socket is then welded to the plate-shaped contact 214 in a flush connection. The opposite end of the tube is crimped onto the proximal end of an electrical conductor 218 which extends distally along the lead 200 to a corresponding electrode 203A, 203B that is configured for contact with body tissue, for example, the heart H (FIG. 4B).

Further, the two-hole electrical contact 214 has a non-active hole 220 that is laterally spaced from the active hole 216 so that the holes 216, 220 are aligned perpendicular to the longitudinal axis of the lead 200. The non-active hole 220 does not support a tubular pin socket.

This alternating construction of a first tubular pin-socket carrier 208 connected to a single-hole electrical contact 212, polymeric insulator ring 210, second tubular pin-socket carrier 208 connected to a two-hole electrical contact 214, polymeric insulator ring 210, third tubular pin-socket carrier 208 connected to a single-hole electrical contact 212, insulator ring 210, fourth tubular pin-socket carrier 208 connected to a two-hole electrical contact 214, insulator ring 210, fifth tubular pin-socket carrier 208 connected to a single-hole contact 212, all supported on the cylindrically-shaped, semi-rigid lead body 206, continues in a distal direction along the lead 200 for as many as there are active terminal pins 50 and side-by-side active terminal pin 50/non-active terminal pin 52 pairs in the feedthrough 40. In total, there are six active terminal pins 50, each followed by a side-by-side pair of an active terminal pin 50/non-active terminal pin 52. However, depending on the medical application that the AMD 12 and lead 200 are designed to treat, there can be less than or greater than that number of an active terminal pin 50 followed by an active terminal pin 50/non-active terminal pin 52 pair as shown in the drawings.

After the alternating series of a tubular pin-socket carrier 208 connected to a single-hole electrical contact 212, polymeric insulator ring 210, tubular pin-socket carrier 208 connected to a two-hole electrical contact 214, polymeric insulator ring 210 is constructed to provide the desired number of tubular pin sockets corresponding to the number of active terminal pins 50 followed by an active terminal pin 50/non-active terminal pin 52 pair comprising the feedthrough 40, the tubular pin-socket carriers and polymeric insulator rings supported on the cylindrically-shaped, semi-rigid lead body 206 as an assembly is over molded with a polymeric material 222, preferably a polyurethane material. This completes construction of the leads 200 shown in FIGS. 7A and 7B.

In another embodiment of the present AMD, there is only one row of a tubular pin-socket carrier 208 connected to a single-hole electrical contact 212 followed by a tubular pin-socket carrier 208 connected to a two-hole electrical contact 214. Moreover, in another embodiment of the present AMD where the non-active terminal pins do not exist, two-hole electrical contact 214 also do not exist. Instead, there are two side-by-side rows of a tubular pin-socket carrier 208 connected to a single-hole electrical contact 212. Further, in still another embodiment according to the present invention, there is only one row of tubular pin-socket carriers 208 connected to a respective single-hole electrical contact 212.

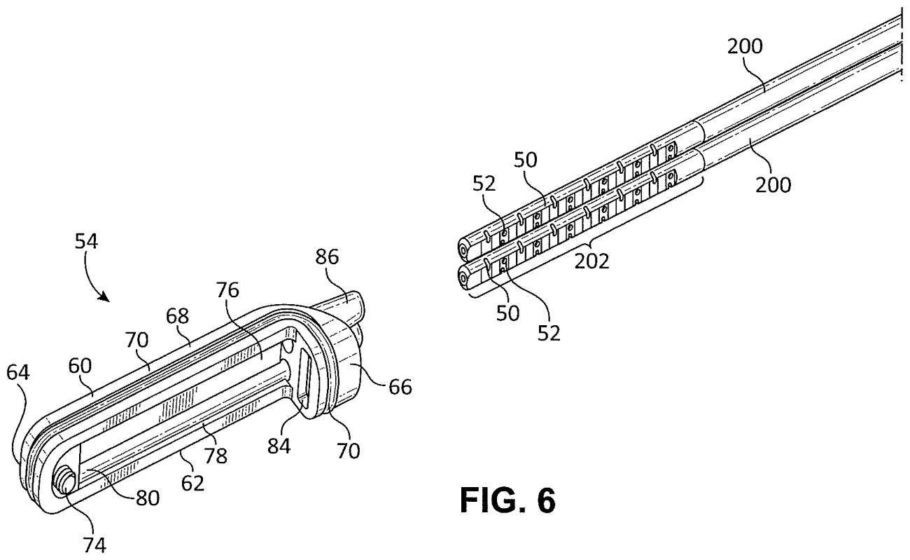

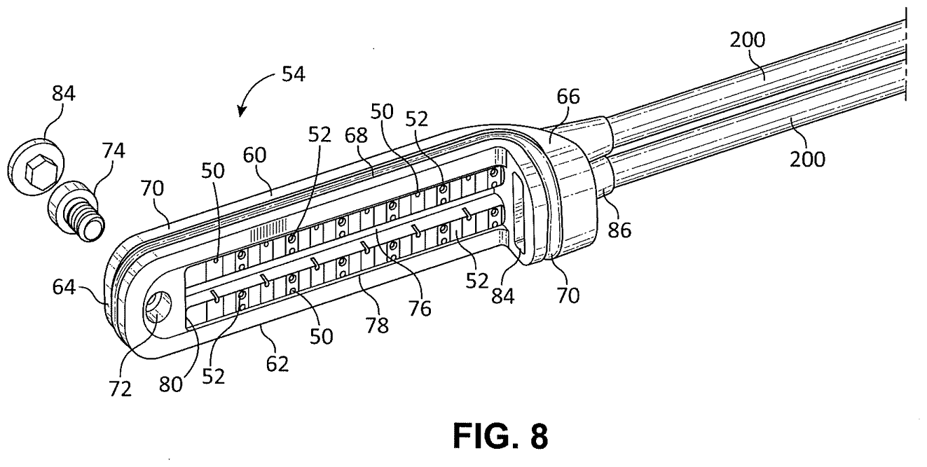

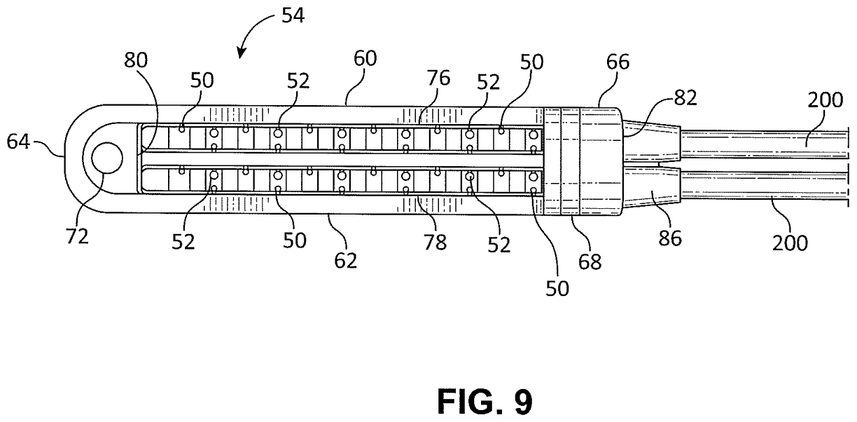

As shown in FIG. 6, the proximal electrical contact portions 202 of the side-by-side leads 200 are then moved through the openings in the depending proximal portion 66 of the polymeric cover 54 and into the respective axial recesses 76, 78 until their proximal ends abut the lead tip datum 80 in the cover. FIGS. 8 and 9 show the proximal electrical contact portions 202 of the leads 200 fully received in the polymeric cover 54 with their respective single-hole electrical contacts 212 and two-hole electrical contacts 214 facing downwardly. The polymeric cover 54 with the side-by-side leads 200 as an assembly in then moved into the recess formed by the planar upper wall 44 of the feedthrough ferrule 42 being spaced below the generally planar upper wall 32 of the lid 28 until the rectangularly-shaped recess 84 in the depending proximal cover portion 66 mates with the rectangularly-shaped locking tab 48 extending from the proximal end wall 46D of the ferrule 42. The threaded opening 72 in the cover 54 is then aligned with the threaded hole 49 residing adjacent to the curved distal inner sidewall portion 38C of the lid 28. A threaded fastener 74 is screwed into the opening 72 to secure this connection. A septum plug 84 (FIGS. 4A and 8) is seated on top of the head of the fastener 74 to help seal the threaded hole 49 and to provide an aesthetic contour to the polymeric cover 54 connected to the AMD 12. The septum plug 84 also helps prevent fibrosis growth of tissue into the hexagonal pocket of the threaded fastener 74. Otherwise, fibrosis growth of tissue could make it difficult to remove the fastener 74.

In that manner, electrical continuity is established from the active terminal pins 50 having their proximal ends connected to the at least two electronic components supported on the previously described PCB assembly (not shown) housed inside the medical device housing comprising the main housing 26 closed by the lid 28. The outwardly extending distal ends of the active terminal pins 50 are received in the metallic tubular pin-sockets supported by the single-hole electrical contacts 212 and the two-hole electrical contacts 214. As previously described, the respective tubular pin-sockets are electrically connected to an electrical conductor 218 which extends distally along the lead to a corresponding electrode 203A, 203B that is configured for contact with body tissue, for example, the heart H (FIG. 4B). Further, the outwardly extending distal ends of the non-active terminal pins 52 received in the corresponding non-active terminal hole 220 of the two-hole electrical contact 214 prevents the proximal electrical contact portions 202 of the leads 200 from being able to move along the axial recesses 76, 78 in the cover 54. The AMD 12 connected to the side-by-side leads 200 is now ready for use in a surgical procedure.

It is appreciated that various modifications to the inventive concepts described herein may be apparent to those skilled in the art without departing from the spirit and scope of the present invention as defined by the hereinafter appended claims.

What is claimed is:

1. An active medical device (AMD) assembly, comprising:
a) an AMD, comprising:
i) a device housing comprising a main housing having a housing sidewall extending to a housing annular edge surrounding a housing opening leading into an interior of the main housing, and a lid connected to the housing annular edge to close the main housing, wherein the lid has a lid upper surface and a lid opening;
ii) a printed circuit board (PCB) assembly contained in the interior of the device housing;
iii) an electrical power source contained in the interior of the device housing to power the PCB assembly; and
iv) a feedthrough, comprising:
A) a ferrule having a ferrule upper surface and a ferrule opening;
B) an insulator brazed into the ferrule opening, wherein the insulator has a thickness that extends from an insulator device side facing the interior of the device housing to an opposed insulator body fluid side;
C) a plurality of first via holes extending through the insulator thickness; and
D) a plurality of first active terminal pins brazed into a respective one of the first via holes in the insulator, wherein the first active terminal pins have a first active terminal pin device portion connected to the PCB assembly and a first active terminal pin body fluid portion extending outwardly beyond the body fluid side of the insulator,
E) wherein the feedthrough is connected to the lid in the lid opening with the ferrule upper surface being spaced below the lid upper surface to thereby provide a lid recess; and
b) a cover that is detachably connectable to the device housing, the cover comprising:
i) a main cover portion having a cover upper surface spaced from a cover lower surface; and
ii) a first axial recess extending along the cover lower surface;
c) a first lead extending distally from a first proximal electrical contact portion to at least two first distal electrodes configured to contact body tissue, the first proximal electrical contact portion comprising a first M number of polymeric carriers supporting a first electrical contact and contacting a first N number of lead insulators in an alternating sequence extending distally from a proximal end of the first proximal electrical contact portion of the first lead, the alternating, distally extending sequence comprising a first of the first N number of lead insulators contacting a first of the first M number of carriers supporting a first electrical contact, a second of the first N number of lead insulators contacting the first of the first M number of carriers, a second of the first M number of carriers supporting a first electrical contact and contacting the second of the first N number of lead insulators, a third of the first N number of lead insulators contacting the second of the first M number of carriers supporting a first electrical contact, a third of the first M number of carriers supporting a first electrical contact and contacting the third of the first N number of lead insulators, a fourth of the first N number of lead insulators contacting the third of the first M number of carriers supporting a first electrical contact, a fourth of the first M number of carriers supporting a first electrical contact and contacting the fourth of the first N number of lead insulators, a fifth of the first N number of lead insulators contacting the fourth of the first M number of carriers supporting a first electrical contact and continuing distally to a distal-most one of the first M number of carriers supporting a first electrical contact and distally contacting a distal-most one of the first N number of lead insulators, wherein each of the first electrical contacts supported by a respective one of the first M number of carriers is connected to a first electrical conductor that extends to a first distal electrode of the first lead,
d) wherein, with the first proximal electrical contact portion of the first lead received in the first axial recess of the cover and with the cover received in the lid recess and having the first electrical contacts facing the first active terminal pins, the first electrical contacts supported by the respective first M number of carriers are electrically connected to a respective one of the first active terminal pins of the feedthrough to thereby establish electrical continuity from the PCB assembly connected to the first active terminal pins connected to the first electrical contacts connected to a respective first electrical conductor connected to a first distal electrode of the first lead.

2. The AMD assembly of claim 1, wherein the first electrical contacts supported by a respective one of the first M number of carriers has an active opening that receives a respective first active terminal pin to thereby establish electrical continuity from the PCB assembly to the first electrical contacts.

3. The AMD assembly of claim 1, further including:
a) a plurality of second via holes extending through the insulator thickness, the second via holes being aligned side-by-side with the first via holes;
b) a plurality of second active terminal pins brazed into a respective one of the second via holes in the insulator, wherein the second active terminal pins have a second active terminal pin device portion connected to the PCB assembly and a second active terminal pin body fluid portion extending outwardly beyond the body fluid side of the insulator, the second active terminal pins being aligned side-by-side with the first active terminal pins in the insulator;
c) the cover further comprising a second axial recess extending along the cover lower surface, the first and second axial recesses being aligned side-by-side in the cover lower surface;
d) a second lead extending distally from a second proximal electrical contact portion to at least two second distal electrodes configured to contact body tissue, the second proximal electrical contact portion comprising a second M number of polymeric carriers supporting a respective second electrical contact and contacting a second N number of lead insulators in an alternating sequence extending distally from a proximal end of the second proximal electrical contact portion of the second lead, the alternating, distally extending sequence comprising a first of the second N number of lead insulators contacting a first of the second M number of carriers supporting a second electrical contact, a second of the second N number of lead insulators contacting the first of the second M number of carriers, a second of the second M number of carriers supporting a second electrical contact and contacting the second of the second N number of lead insulators, a third of the second N number of lead insulators contacting the second of the second M number of carriers supporting a second electrical contact, a third of the second M number of carriers supporting a second electrical contact and contacting the third of the second N number of lead insulators, a fourth of the second N number of lead insulators contacting the third of the second M number of carriers supporting a second electrical contact, a fourth of the second M number of carriers supporting a second electrical contact and contacting the fourth of the second N number of lead insulators, a fifth of the second N number of lead insulators contacting the fourth of the second M number of carriers supporting a second electrical contact and continuing distally to a distal-most one of the second M number of carriers supporting a second electrical contact and distally contacting a distal-most one of the second N number of lead insulators, wherein each of the second electrical contacts supported by a respective one of the second M number of carriers is connected to a second electrical conductor that extends to a second distal electrode of the second lead,
e) wherein, with the second proximal electrical contact portion of the second lead received in the second axial recess of the cover and with the cover received in the lid recess and having the second electrical contacts facing the second active terminal pins, the second electrical contacts supported by the respective second M number of carriers are electrically connected to a respective one of the second active terminal pins of the feedthrough to thereby establish electrical continuity from the PCB assembly connected to the second active terminal pins connected to the second electrical contacts connected to a respective second electrical conductor connected to a second distal electrode of the second lead.

4. The AMD assembly of claim 1, wherein the insulator of the feedthrough further has a plurality of first non-active terminal pins brazed into a respective one of a plurality of first non-active via holes, the first non-active terminal pins having a first non-active body fluid portion that extends outwardly beyond the body fluid side of the insulator, and wherein the first electrical contacts supported by the second, fourth, and continuing distally for every even number of the first M number of carriers also has a non-active opening which receives a first non-active body fluid portion of a respective first non-active terminal pin.

5. The AMD assembly of claim 1, wherein the insulator of the feedthrough further has a plurality of first non-active terminal pins brazed into a respective one of a plurality of first non-active via holes, the first non-active terminal pins having a first non-active body fluid portion that extends outwardly beyond the body fluid side of the insulator, and wherein the first electrical contacts supported by the first, third, and continuing distally for every odd number of the first M number of carriers also has a non-active opening which receives a first non-active body fluid portion of a respective first non-active terminal pin.

6. The AMD assembly of claim 3, wherein the main cover portion extends to a depending cover portion, and wherein the main cover portion extends along a primary cover axis from a distal cover end wall to the depending proximal cover portion aligned along a secondary cover axis that is substantially perpendicular to the primary cover axis.

7. The AMD assembly of claim 6, wherein the main cover portion extending to the depending cover portion has a cover upper surface spaced from a cover lower surface with the cover upper and lower surfaces extending to a cover front sidewall opposite a cover back sidewall, and wherein an endless groove residing in the front and back sidewalls, the curved distal end wall and the depending proximal curved portion supports a pliable endless ring-shaped sealing gasket.

8. The AMD assembly of claim 6, wherein the depending proximal cover portion is disposed adjacent to the housing sidewall.

9. The AMD assembly of claim 6, wherein the first and second axial recesses extending along the cover lower surface extend through the depending proximal cover portion to an outer surface of the depending cover portion.

10. The AMD assembly of claim 4, wherein the plurality of non-active terminal pins brazed into a respective one of the plurality of first non-active via holes do not extend through the insulator thickness into the interior of the main housing.

11. The AMD assembly of claim 4, wherein the plurality of first active terminal pins have a first diameter perpendicular to their first longitudinal axis and the plurality of first non-active terminal pins have a second diameter perpendicular to their second longitudinal axis, and wherein the second diameter of the first non-active terminal pins is equal to, less than or greater than the first diameter of the first active terminal pins.

12. The AMD assembly of claim 6, wherein the ferrule opening is oval-shaped comprising a proximal curved end opposite a distal curved end, and the ferrule of the feedthrough has a threaded opening adjacent to the proximal curved end and an outwardly extending locking tab adjacent to the distal curved end of the oval-shaped ferrule opening, and wherein the cover has a threaded opening adjacent to the distal cover end wall and a locking recess located in the depending proximal cover portion, and wherein, when the cover is detachably connected to the device housing, the cover threaded opening is aligned with the ferrule threaded opening and the outwardly extending locking tab of the ferrule is received in the locking recess in the depending proximal cover portion so that a fastener threaded through the cover threaded opening aligned with the feedthrough threaded opening connects the cover to the device housing.

13. An active medical device (AMD) assembly, comprising:
a) an AMD, comprising:
i) a device housing comprising a main housing having a housing sidewall extending to a housing annular edge surrounding a housing opening leading into an interior of the main housing, and a lid connected to the housing annular edge to close the main housing, wherein the lid has a lid upper surface and a lid opening;
ii) a printed circuit board (PCB) assembly contained in the interior of the device housing;
iii) an electrical power source contained in the interior of the device housing to power the PCB assembly; and
iv) a feedthrough, comprising:
A) a ferrule having a ferrule upper surface and a ferrule opening;
B) an insulator brazed into the ferrule opening, wherein the insulator has a thickness that extends from an insulator device side facing the interior of the device housing to an opposed insulator body fluid side;
C) a plurality of first via holes extending through the insulator thickness, and a plurality of second via holes extending through the insulator thickness, the second via holes being aligned side-by-side with the first via holes;
D) a plurality of first active terminal pins brazed into a respective one of the first via holes in the insulator, wherein the first active terminal pins have a first active terminal pin device portion connected to the PCB assembly and a first active terminal pin body fluid portion extending outwardly beyond the body fluid side of the insulator, and a plurality of second active terminal pins brazed into a respective one of the second via holes in the insulator, wherein the second active terminal pins have a second active terminal pin device portion connected to the PCB assembly and a second active terminal pin body fluid portion extending outwardly beyond the body fluid side of the insulator, the second active terminal pins being aligned side-by-side with the first active terminal pins in the insulator; and
E) wherein the feedthrough is connected to the lid in the lid opening with the ferrule upper surface being spaced below the lid upper surface to thereby provide a lid recess;
b) a cover that is detachably connectable to the device housing, the cover comprising:
i) a main cover portion extending along a primary cover axis from a distal cover end wall to a depending proximal cover portion aligned along a secondary cover axis that is substantially perpendicular to the primary cover axis, wherein the main cover portion has a cover upper surface spaced from a cover lower surface with the cover upper and lower surfaces extending to a cover front sidewall opposite a cover back sidewall; and
ii) a first axial recess and a second axial recess extending along the cover lower surface and through the depending proximal cover portion, the first and second axial recesses being aligned side-by-side in the cover lower surface;
c) a first lead extending distally from a first proximal electrical contact portion to at least two distal electrodes configured to contact body tissue, the first proximal electrical contact portion comprising a first M number of polymeric carriers supporting an electrical contact and contacting a first N number of insulators in an alternating sequence extending distally from a proximal end of the first proximal electrical contact portion of the first lead, the alternating, distally extending sequence comprising a first of the first N number of insulators contacting a first of the first M number of carriers supporting an electrical contact, a second of the first N number of insulators contacting the first of the first M number of carriers, a second of the first M number of carriers supporting an electrical contact and contacting the second of the first N number of insulators, a third of the first N number of insulators contacting the second of the first M number of carriers supporting an electrical contact, a third of the first M number of carriers supporting an electrical contact and contacting the third of the first N number of insulators, a fourth of the first N number of insulators contacting the third of the first M number of carriers supporting an electrical contact, a fourth of the first M number of carriers supporting an electrical contact and contacting the fourth of the first N number of insulators, a fifth of the first N number of insulators contacting the fourth of the first M number of carriers supporting an electrical contact and continuing distally to a distal-most one of the first M number of carriers supporting an electrical contact and distally contacting a distal-most one of the first N number of insulators, wherein each of the first electrical contacts supported by the first M number of carriers is connected to an electrical conductor that extends to a distal electrode of the first lead,
d) a second lead extending distally from a second proximal electrical contact portion to at least two second distal electrodes configured to contact body tissue, the second proximal electrical contact portion comprising a second M number of polymeric carriers supporting a respective second electrical contact and contacting a second N number of lead insulators in an alternating sequence extending distally from a proximal end of the second proximal electrical contact portion of the second lead, the alternating, distally extending sequence comprising a first of the second N number of lead insulators contacting a first of the second M number of carriers supporting a second electrical contact, a second of the second N number of lead insulators contacting the first of the second M number of carriers, a second of the second M number of carriers supporting a second electrical contact and contacting the second of the second N number of lead insulators, a third of the second N number of lead insulators contacting the second of the second M number of carriers supporting a second electrical contact, a third of the second M number of carriers supporting a second electrical contact and contacting the third of the second N number of lead insulators, a fourth of the second N number of lead insulators contacting the third of the second M number of carriers supporting a second electrical contact, a fourth of the second M number of carriers supporting a second electrical contact and contacting the fourth of the second N number of lead insulators, a fifth of the second N number of lead insulators contacting the fourth of the second M number of carriers supporting a second electrical contact and continuing distally to a distal-most one of the second M number of carriers supporting a second electrical contact and distally contacting a distal-most one of the second N number of lead insulators, wherein each of the second electrical contacts supported by a respective one of the second M number of carriers is connected to a second electrical conductor that extends to a second distal electrode of the second lead,
e) wherein, with the first and second proximal electrical contact portions of the respective first and second leads received in the respective first and second axial recesses of the cover and with the cover received in the lid recess and having the first electrical contacts facing the first active terminal pins and the second electrical contacts facing the second active terminal pins, the first electrical contacts supported by the respective M number of the first carriers are electrically connected to a respective one of the first active terminal pins and the second electrical contacts supported by the respective M number of the second carriers are electrically connected to a respective one of the second active terminal pins of the feedthrough to thereby establish electrical continuity from the PCB assembly connected to the first and second active terminal pins connected to the first and second electrical contacts connected to a respective electrical conductor connected to a distal electrode of the respective first and second leads.

14. The AMD assembly of claim 13, wherein the insulator of the feedthrough further comprises:
a) a plurality of first non-active terminal pins brazed into a respective one of a plurality of first non-active via holes, the first non-active terminal pins having a first non-active body fluid portion that extends outwardly beyond the body fluid side of the insulator, and wherein the first electrical contacts supported by the second, fourth, and continuing distally for every even number of the first M number of carriers also has a non-active opening which receives a first non-active body fluid portion of a respective first non-active terminal pin, and wherein the first active and non-active terminal pins extend side-by-side in the insulator; and
b) a plurality of second non-active terminal pins brazed into a respective one of a plurality of second non-active via holes, the second non-active terminal pins having a second non-active body fluid portion that extends outwardly beyond the body fluid side of the insulator, and wherein the second electrical contacts supported by the second, fourth, and continuing distally for every even number of the second M number of carriers also has a non-active opening which receives a second non-active body fluid portion of a respective second non-active terminal pin, and wherein the second active and non-active terminal pins extend side-by-side in the insulator.

15. The AMD assembly of claim 13, wherein the insulator of the feedthrough further comprises:
a) a plurality of first non-active terminal pins brazed into a respective one of a plurality of first non-active via holes, the first non-active terminal pins having a first non-active body fluid portion that extends outwardly beyond the body fluid side of the insulator, and wherein the first electrical contacts supported by the first, third, and continuing distally for every odd number of the first M number of carriers also has a non-active opening which receives a first non-active body fluid portion of a respective first non-active terminal pin, and wherein the first active and non-active terminal pins extend side-by-side in the insulator; and
b) a plurality of second non-active terminal pins brazed into a respective one of a plurality of second non-active via holes, the second non-active terminal pins having a second non-active body fluid portion that extends outwardly beyond the body fluid side of the insulator, and wherein the second electrical contacts supported by the first, third, and continuing distally for every odd number of the second M number of carriers also has a non-active opening which receives a second non-active body fluid portion of a respective second non-active terminal pin, and wherein the second active and non-active terminal pins extend side-by-side in the insulator.

16. The AMD assembly of claim 13, wherein an endless groove residing in the front and back sidewalls, the curved distal end wall and the depending proximal curved portion supports a pliable endless ring-shaped sealing gasket.

17. The AMD assembly of claim 13, wherein the first and second axial recesses extending along the cover lower surface extend through the depending proximal cover portion to an outer surface of the depending cover portion.

18. The AMD assembly of claim 13, wherein the plurality of non-active terminal pins brazed into a respective one of the plurality of first non-active via holes do not extend through the insulator thickness into the interior of the main housing.

19. The AMD assembly of claim 13, wherein the plurality of first active terminal pins have a first diameter perpendicular to their first longitudinal axis and the plurality of first non-active terminal pins have a second diameter perpendicular to their second longitudinal axis, and wherein the second diameter of the first non-active terminal pins is equal to, less than or greater than the first diameter of the first active terminal pins.

20. The AMD assembly of claim 13, wherein the plurality of first non-active terminal pins brazed into a respective one of the plurality of first non-active via holes and the plurality of second non-active terminal pins brazed into a respective one of the plurality of second non-active via holes do not extend through the insulator thickness into the interior of the main housing.
